(19) United States
(12) Patent Application Publication
(10) Pub. No.: US 2004/0046934 A1
(43) Pub. Date: Mar. 11, 2004

(54) Oculokinetic offset acuity testing

(75) Inventors: William E. Sponsel, San Antonio, TX (US); Christina Bui, Nashville, TN (US); John Simms, Dallas, TX (US); G. L. Mayo, Tucson, AZ (US); K. J. Frasier, Lubbock, TX (US)

Correspondence Address: Michael C. Barrett, Esq., FULBRIGHT & JAWORSKI, L.L.P., Suite 2400, 600 Congress Avenue, Austin, TX, 78701, US

(73) Assignee: Board of Regents, The University of Texas System

(21) Appl. No.: 10/366,588

(22) Filed: Feb. 13, 2003

Related U.S. Application Data
(60) Provisional application No. 60/356,683, filed on Feb. 14, 2002.
(60) Provisional application No. 60/358,518, filed on Feb. 19, 2002.

(51) Int. Cl. A61B 3/00

(52) U.S. Cl. 351/200

(57) ABSTRACT

Methods and apparatuses for measuring visual acuity. In one embodiment, a moving fixation target is displayed. A series of optotype letters appearing offset from the moving fixation target is also displayed, and each optotype letter corresponds to a visual acuity. Each optotype letter appears in one of four possible random quadrant locations adjacent the moving fixation target, and each optotype appears only for a limited time.

This application claims priority to, and incorporates by reference, the following two U.S. Provisional Patent Applications: 1) U.S. Provisional Patent Application Serial No. 60/356,683 (filed Feb. 14, 2002) and 2) U.S. Provisional Patent Application Serial No. 60/358,518 (filed Feb. 19, 2002).

### BACKGROUND OF THE INVENTION

1. Field of the Invention

The present invention relates generally to visual testing. More particularly, it concerns methods and apparatuses for measuring visual acuity of a patient. Even more particularly, it concerns, in one embodiment, measuring such acuity by combining an oculokinetic fixation target with eye-chart letter stimuli under time limited viewing conditions.

2. Description of Related Art

Three prevalent causes of vision loss are macular degeneration, glaucoma, and diabetic retinopathy.

Macular Degeneration is a progressive and incurable disease of the retina wherein the light-sensing cells in the central area of vision (the macula) stop working and eventually die. The disease is most common in people who are age sixty and over, and for this reason it is often called age-related macular degeneration (AMD). About 10% of macular degeneration cases are the “wet” (or “exudative”) form, in which newly-formed, immature blood vessels grow from the choroid and leak into the spaces above and below the retinal pigment epithelium (RPE). This leakage (neovascularization) can damage the photoreceptor cells and cause permanent central vision loss. Most cases of macular degeneration are the “dry,” (or “atrophic”) form, distinguished by yellowish deposits of debris in the retina (specifically, Bruch's membrane). Called “drusen,” the material comprising these deposits is usually carried away by the same blood vessels which bring nutrients to the retina. For reasons yet unknown, this process is diminished in macular degeneration. Although it rarely results in complete blindness, macular degeneration typically robs an individual of all but the outermost, peripheral vision, leaving only dim images or black holes at the center of vision. As the population ages, and the “baby boomers” advance into their 50's and 60's, it is expected that the United States will see a virtual epidemic of AMD. Perhaps 14%-24% of the U.S. population aged 65-74 years and 35% of people aged 75 years or more have the disease.

Other less common types of macular degeneration, which are hereditary and can affect younger people, are Best's disease, Stargardt's disease, and Sorsby's disease. Collectively, these types are called juvenile macular degeneration.

The glaucomas are a series of progressively acting eye diseases that can ultimately lead to blindness. Around the globe, the number of people with glaucoma has been expected to exceed 66 million in the early 2000's. Due to its prevalence, the World Health Organization estimates that the glaucomas may be the most common world-wide cause of irreversible blindness. Unlike some eye disorders, the causes of the glaucomas and the best way to treat them are unclear. The diseases are often, but not always, associated with elevated intraocular pressure (IOP) as a result of reduced drainage of aqueous humour, the fluid that fills the anterior chamber of the eye in front of the lens. This elevated pressure may damage delicate nerve fibres that, via the optic nerve, carry visual signals from the retina to the brain.

Diabetic retinopathy damages tiny blood vessels that supply the retina. In the early stages of this disease, called non-proliferative or “background” retinopathy, the retinal vessels weaken and develop bulges (microaneurysms) that may leak blood (hemorrhages) or fluid (exudates) into the surrounding tissue. Vision is rarely affected during this stage of retinopathy. If proliferative retinopathy is left untreated, about half of those who have it will become blind within five years, compared to just 5% of those who receive treatment. Proliferative retinopathy, a later stage of the disease, involves the growth of fragile new blood vessels on the retina and into the vitreous—a jelly-like substance inside the eye. These vessels can rupture and release blood into the vitreous, which causes blurred vision or temporary blindness. The scar tissue that may subsequently develop can pull on the retina and cause retinal detachment, which may lead to permanent vision loss. Macular edema may also occur.

To detect the loss of visual acuity that results from these, and other, serious disorders, patients and clinicians most typically turn to standard eye chart tests, Amsler grid tests, opthalmoscopy, fundus photography, and fluorescein angiogram (FA). Although useful to a degree, these measurement techniques, however, exhibit serious shortcomings. For instance, some of this conventional technology may fail to recognize a patient who exhibits warning signs of serious eye disorders. Further, some of this conventional technology may involve complicated testing that requires a skilled practitioner to interpret the results. Still further, none of this conventional technology is well-suited for performing adequate visual acuity testing currently required by the Food and Drug Administration (FDA) for certain investigational new drug applications.

Accordingly, there is a need for new, improved techniques for measuring visual acuity.

The referenced shortcomings of conventional methodologies mentioned above are not intended to be exhaustive, but rather are among many that tend to impair the effectiveness of previously known techniques concerning the laser treatment of cutaneous vascular lesions. Other noteworthy problems may also exist; however, those mentioned here are sufficient to demonstrate that methodology appearing in the art have not been altogether satisfactory and that a significant need exists for the techniques described and claimed herein.

### SUMMARY OF THE INVENTION

Shortcomings of the prior art are reduced or eliminated by the techniques disclosed herein. These techniques are applicable to a vast number of applications, including but not limited to applications involving clinical testing of patient's visual acuity and testing in order to meet specific FDA guidelines.

The inventors have discovered that, although patients may be suffering from a significant loss in visual acuity, those patient may nevertheless “pass” several visual acuity tests. Without being bound by theory, it is believed that small-movements (microsaccades) allow such patients, even if they have large perifoveolar and macular defects, to “fill-in” scotomata and achieve normal vision results on standard acuity or Amsler testing.

With the techniques of this disclosure, however, the vision loss of such patients may nevertheless be identified. This is done, in one embodiment, by subjecting those patients to a visual acuity test using Oculokinetic Offset Acuity (OKAy) Testing. In one embodiment, that testing involves having the patient follow a moving fixation target while identifying optotype letters (such as Early Treatment Diabetic Retinopathy Study (ETDRS) letters) corresponding to various acuities (e.g., 20/20, 20/40, 20/50, 20/70, 20/100, and 20/200) that are displayed for various time durations (e.g., 1 second, 0.5 seconds, 0.2 seconds, and 0.1 seconds) at different, random quadrants relative to the moving fixation target.

The inventors have discovered that the ability to “fill-in” scotomata appears to lapse between about 0.2 and about 0.1 seconds when subjected to the presentation of optotypes in random quadrants. Thus, if an optotype letter is momentarily displayed for such a time period (or a different, time period suitable to identify visual disorders) in a random quadrant, patients exhibiting scotomata will fail to identify the letter although, given more time, they may be able to eventually make the identification.

Using this type of testing allows practitioners to not only accurately assess visual acuity, but also to quickly and inexpensively show compliance with current FDA guidelines concerning visual acuity measurements. Exhibiting compliance with these regulations, in turn, may lead to quicker commercialization of various new eye treatments.

In one embodiment, the invention involves an apparatus for measuring visual acuity including a display configured to show a moving fixation target and a series of optotypes appearing adjacent the moving fixation target, each optotype appearing only for a limited time.

In another embodiment, the invention involves an apparatus for measuring visual acuity including a display configured to show a moving fixation target and a series of optotype letters appearing adjacent the moving fixation target, each optotype letter corresponding to a visual acuity, each optotype letter appearing in one of four possible random quadrant locations adjacent the moving fixation target, and each optotype appearing only for a limited time.

In another embodiment, the invention involves a computer program for measuring visual including instructions for displaying a moving fixation target and instructions for displaying a series of optotypes appearing adjacent the moving fixation target, each optotype appearing only for a limited time.

In another embodiment, the invention is a method for measuring visual acuity. A moving fixation target is displayed, and a series of optotypes appearing adjacent the moving fixation target are displayed, each optotype appearing only for a limited time.

In another embodiment, the invention is a method for measuring visual acuity. A moving fixation target is displayed. Further, a series of optotype letters appearing adjacent the moving fixation target is displayed, each optotype letter corresponding to a visual acuity, each optotype letter appearing in one of four possible random quadrant locations adjacent the moving fixation target, and each optotype appearing only for a limited time.

Other features and associated advantages will become apparent with reference to the following detailed description of specific embodiments in connection with the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a prior art Snellen eye-chart illustrating a conventional technique for measuring visual acuity.

FIG. 2 is a schematic diagram illustrating an opening screen of a visual acuity test in accordance with embodiments of the present disclosure.

FIG. 3 is a schematic diagram illustrating a visual acuity test using optotype letters corresponding to a first visual acuity (i.e., letters of a first, particular size) in accordance with embodiments of the present disclosure.

FIG. 4 is a schematic diagram illustrating a visual acuity test using optotype letters corresponding to a second visual acuity (i.e., letters of a second, particular size) in accordance with embodiments of the present disclosure.

FIG. 5 is a schematic diagram illustrating a visual acuity test using optotype letters corresponding to mixed visual acuities (i.e., letters of a mixed sizes) in accordance with embodiments of the present disclosure.

FIG. 6 is a schematic diagram illustrating four quadrants within which an optotype may be placed in accordance with embodiments of the present disclosure.

FIG. 7 is a schematic diagram illustrating the motion and display times associated with a visual acuity test using optotype letters, in accordance with embodiments of the present disclosure.

### DESCRIPTION OF ILLUSTRATIVE EMBODIMENTS

Each of U.S. Pat. Nos. 6,313,155; 6,046,223; and 5,789,435 is hereby incorporated by reference in its entirety.

The present disclosure describes techniques for measuring visual acuity so that vision loss that would go undetected using conventional tests may be readily identified. In particular, these techniques advantageously provide a rapid test for detecting occult pericentral scotomata. Thus, these techniques may be invaluable for improving the ability to identify macular degeneration, glaucoma, and/or diabetic retinopathy—the three current major causes of acquired blindness—in patients. These techniques may greatly benefit practitioners working in a clinical setting. Further, these techniques have a strong potential for use in new drug studies. Specifically, given the limited scope of FDA approved endpoints for therapeutic efficacy, these techniques may be invaluable for helping show compliance with FDA guidelines.

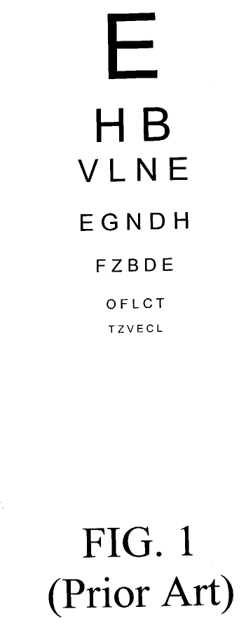

Turning first to FIG. 1, there is shown a prior art Snellen eye-chart.

As illustrated, the eye-chart is made up of different lines of optotype letters (these optotype letters may be referred to by some practitioners as ETDRS letters). Each different line of the eye-chart includes optotype letters of a different size. The patient views these different-sized optotype letters from a fixed distance. Accordingly, the different-sized letters may be made to correspond to different visual acuities. In particular, one line of the chart may correspond to 20/20, while other larger typefaces may correspond to acuities of, for instance, 20/40, 20/50, 20/70, 20/100, or 20/200. As is known in the art, an eye-chart such as the one shown in FIG. 1 may be projected or be displayed using one or more mirrors to simulate a particular working distance from the patient.

The prior art eye-chart of FIG. 1 is widely used to identify patients with vision loss, and particularly, pericentral scotomata. Although useful at least to some degree, the inventors have discovered that patients with even large perifoveolar and macular defects may “fill-in” scotomata and achieve normal vision results (i.e., around 20/20 acuity) using this standard type of test. Without being bound by theory, this “filling-in” is believed to come about by small-movements (microsaccades) of the eye. These microsaccades allow “good” portions of a patient's eye to compensate for portions exhibiting scotoma. Roughly speaking, this phenomena may be imagined as normal portions of the eye quickly scanning over a blind spot so that the patient does not even realize that he/she has the blind spot. Using microsaccades, a patient exhibiting even several blind-spots may nevertheless fill-in missing parts of the eye chart unconsciously and appear to the practitioner to have 20/20 vision. This patient, unfortunately, will not receive the early treatment he or she may require to alleviate the problem.

The inventors have discovered that the standard eye chart of FIG. 1 lends itself to this type of deceiving, microsaccade-induced result, in part, because the patient is usually given unlimited time to discern a particular letter on the eye chart. Given ample time, the eye of the patient does what has become natural to it—it uses microsaccades (and corresponding mental ability in reconstructing missing scenes) to “fill-in” blind spots. The inventors have noticed that this “filling-in” process works better in some patients than others, as is the case that some people may be better at seeing only one part of a scene and mentally reconstructing the remaining portions.

The microsaccades discussed above may not only thwart a patient's need to receive early treatment by apparently passing an acuity test, but they also may thwart drug companies from showing compliance with current FDA guidelines. As an example, the FDA currently sets endpoints for some clinical trials based upon visual acuity (e.g., endpoints related to glaucoma treatments). In particular, the FDA considers the doubling of visual angle to be clinically significant. The FDA informs that the doubling of visual angle is equivalent to three lines on an ETDRS chart (i.e., a chart such as the one shown in FIG. 1). According to the FDA, this doubling of visual angle may be represented as a percentage of patients with a doubling of visual angle or as a mean change of three lines or more. Due to at least the microsaccade-phenomenon discussed above, however, a mean change in 3 lines on a chart such as the chart of FIG. 1 patients may be impossible to show, even if a new drug is indeed effectively treating the eyes of a patient. In particular, the patient before the drug treatment may be able to (incorrectly) show a 20/20 acuity through the use of microsaccades. Thus, although the new drug may have eliminated or reduced scotomata, that patient would keep scoring 20/20 on the eye chart. Hence, a drug company may not be able to show compliance with the visual acuity portion of the FDA guideline despite the fact that a drug is indeed being effective.

In view of at least the above, the inventors realized that a better visual acuity test was needed—one that would identify patients as having lost visual acuity even if they were skilled in the art of microsaccades that could “fool” standard eye tests. With such a visual acuity test, patients in need of treatment could be identified much earlier than they would be with standard tests. Further, drug companies could show compliance with FDA guidelines because, for instance, a change in three lines would actually be measurable because patients would no longer be scoring false, perfect acuities brought about by microsaccades.

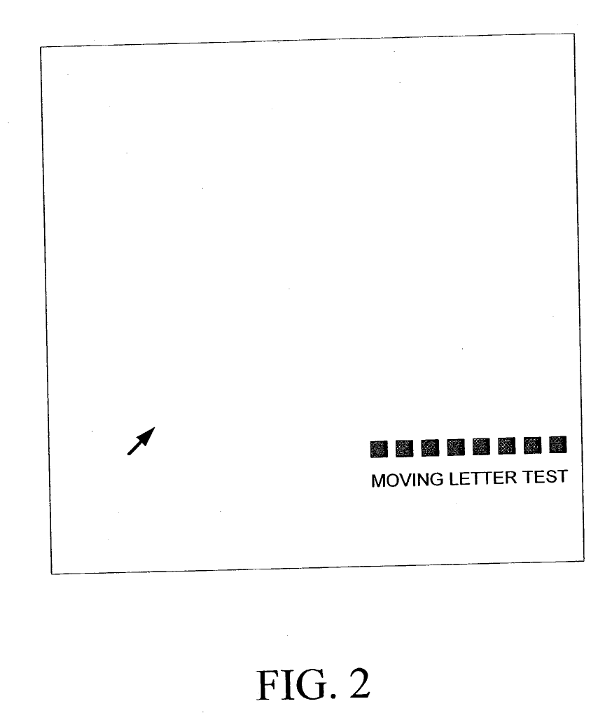

FIGS. 2-7 show embodiments of the present disclosure directed to techniques for a new type of visual testing scheme that measures visual acuity and detects pathology leading to, for instance, pericentral visual field loss. These techniques can detect visual defects that elude standard testing protocols and can correspondingly aid drug companies determine the efficacy of new drugs, using the guidelines already set forth by the FDA.

The inventors have termed embodiments of their testing techniques Oculokinetic Offset Acuity (OKAy) testing. Generally speaking, preferred embodiments of this testing scheme involve the display of a moving fixation target coupled with a time-limited presentation of ETDRS optotype letters offset from the fixation target in one of four quadrants. In operation, the patient follows the moving fixation target and identifies the letters he or she sees that appear next to the moving fixation target. Because the letters may be made to correspond to visual acuities, this testing determines visual acuity. Further, because the presentation of the letters may be relatively quick (and placed in a random quadrant relative to the fixation target), the patient does not have time to “fill-in” visual defects by way of microsaccades. Thus, the techniques is also able to detect occult defects that would go unnoticed through standard testing. Accordingly, the testing techniques of this disclosure reveal two types of visual acuity—one owing to the ability to read optotype letters of a particular size (this is the type of visual acuity standard eye-charts aim to measure) and another owing to the ability to identify a letter of a particular size without having the benefit of microsaccades. The inventors have coined the second of these two types of visual acuities as being the “actual” visual acuity. It is this “actual” visual acuity that standard techniques cannot measure, and it is exactly this type of visual acuity that is so important for drug companies to be able to measure to show compliance with FDA guidelines.

Having described general aspects and advantages of OKAy testing, it is now appropriate to methodically describe other exemplary, non-limiting OKAy embodiments illustrated in FIGS. 2-7.

Turning next to FIG. 2, there is shown an opening screen of a visual acuity test according to embodiments of this disclosure.

In FIG. 2, the screen signifies to the patient and/or practitioner that the test involves something that can be thought of as a “Moving Letter Test.” Shown is a cursor (the arrow in FIG. 2), which may be used to select one of eight different colored squares (the squares appearing above the text “Moving Letter Test”). In one embodiment, selection of each different square may start an OKAy visual acuity test utilizing optotype letters of a different size (and, hence, corresponding to a different acuity). For instance, by clicking on the left-most square may begin a test in which a moving fixation target would be displayed with intermittent, large ETDRS letters appearing alongside the target. In particular, these large letters may correspond to a first visual acuity, such as 20/200. Clicking on the square immediately to the right of this first square may begin a test involving the next smaller size of optotype letters, corresponding to a different acuity, such as 20/100. This progression may continue until one clicks on the right-most square, which may begin a test involving the smallest size of optotype letters. The smallest size, in different embodiment may be, for instance, 20/20 or 20/15.

Of course, it will be recognized by those having skill in the art, that other opening screens (or no opening screen at all) may be used to practice this invention. Further, in other embodiments, different identifiers may be used to start different tests, and the progression between differently-sized optotypes may be arranged in an order other than largest to smallest. In fact, practitioners may choose to use any type of opening screen to their own liking. Such a screen may be chosen to best convey a particular opening message and/or to display some basic functionality and/or control of the test.

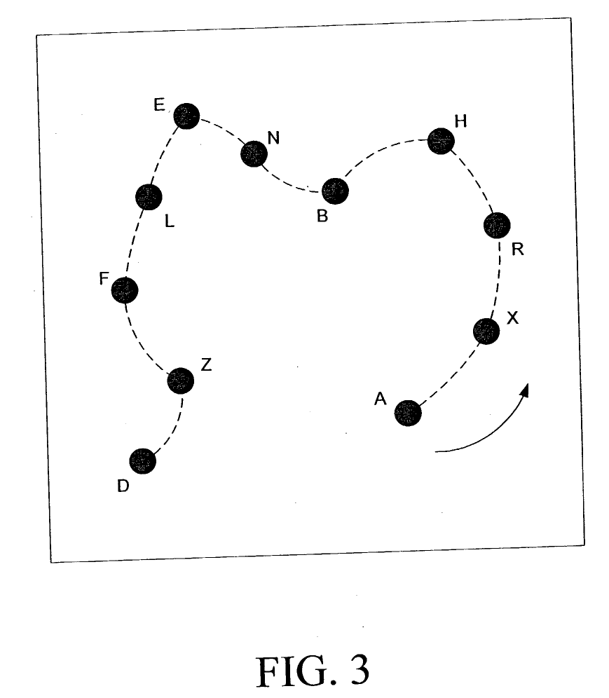

Turning next to FIG. 3, there is a shown a schematic diagram illustrating an embodiment of the OKAy testing scheme.

In FIG. 3, the colored circle represents a moving fixation target. The moving fixation target moves along a path shown by the dashed line (in practice, the dashed line would not typically be shown on a display—otherwise, a patient would be able to “lookahead” and predict where the fixation target was going). In one embodiment, the path taken by the moving fixation target may be a random, continuous path. By “continuous,” it is meant that the moving fixation target would not, for instance, jump from the left most corner of the screen to the right-most corner of the screen; rather, the moving fixation target would move continuously about the screen so that a patient may readily follow it without making drastic, discontinuous eye or head movements. In another embodiment, the moving fixation target may follow some fixed path, such as a spiral or some other pattern.

In one embodiment, the moving fixation target may be a circle (as illustrated), but in other embodiments, any suitable shape and color for fixing the gaze of a patient may be used. In the illustrated embodiment, the moving fixation target is a red circle having a diameter (upon a standard PC laptop monitor) of about 0.5 centimeters. The moving fixation target may make sound(s) while traversing the display or be silent. For younger children, the moving fixation target may be symbols designed to catch their attention—such as brightly-colored cartoon-type character or the like. Likewise, the fixation target may make noises to ensure that the child is paying attention to it. Of course, the type of moving fixation target may be selected by the practitioner through a suitable opening screen and/or by some other control mechanism well known in the art.

As the moving fixation target of FIG. 3 traverses its path about the display, a series of optotypes are intermittently displayed for various time periods adjacent the fixation target. With reference to FIG. 3, upon starting the test, the optotype letter “A” is immediately displayed in the upper-left quadrant adjacent the moving fixation target. The optotype letter “A” will only appear for a limited time. According to different embodiments, this limited time may be between about 0.05 seconds and about 5 seconds. More particularly, it may be between about 0.1 seconds and about 1 second. Even more particularly, it may be about 0.1 seconds, about 0.2 seconds, about 0.5 seconds, or about 1.0 second. Those having skill in the art will recognize, however, that any time period suitable for having a patient identify an optotype may suffice. In this regard, times shorter than 0.05 seconds may be suitable in some circumstances while times greater than 1 second may also suffice.

For the sake of example, assume that the letter “A” appears for 1 second. During this one second, the moving fixation target may pause (i.e., stop its travel while displaying the letter) or continue to move along with the “A” moving along its side, remaining in the upper-left quadrant. Either way, the patient will see the letter “A adjacent the moving fixation target for 1 second. After that one second has elapsed, the letter “A” disappears and the moving fixation target continues moving along its path (now with no letters around it). After some period of time (which may be fixed or variable and in one embodiment may be in a range between about 0.5 and about 20 seconds), the second in the series of optotypes may be displayed. In one embodiment, this time may be based upon the reaction time of the patient—for example, if it is difficult for a patient to keep-up with the test, the time between the display of optotypes may be increased.

In FIG. 3, the second optotype is the optotype letter “X.” The letter “X” is shown here in the upper-right quadrant adjacent the moving fixation target. As the case with the previous letter, the letter “X” is displayed for a limited time. For instance, it too may be displayed for one second, as was the case with the letter “A.” Alternatively, it may be displayed for any other suitable, limited time period. After this time period, the “X” disappears until the third in the series of optotypes is displayed. This time, the optotype is the letter “R,” shown in a lower-right quadrant adjacent the moving fixation target. As the test progresses in FIG. 3, the presentation time for the optotype letters may get progressively smaller. For instance, while the “A” and “X” may be shown for about 1 second, the “F,” “D,” and “Z” may each be shown for about 0.1 seconds. The “B,” “N,” and “E,” on the other hand, may be shown for about 0.5 or 0.2 seconds. Of course, different timing schemes will be apparent to those having skill in the art having the benefit of this disclosure.

This sequence of displaying optotype letters adjacent the moving fixation target in an intermittent (i.e., one letter appears, then disappears, and then later the next letter appears), time-limited (i.e., each letter is shown only for a limited time) manner continues for as long as the test is desired to last. In FIG. 3, the series of optotype letters includes 11 letters. In other embodiments, a different number of course may be used. For instance, a test could include a series of 2, 3, 4, 5, 6, 7, 8, 9, 10, 12, 13, 14, 15, 16, 17, 18, 19, 20, 21, 22, 23, 24, 25, and so on letters. In this regard, even a single optotype being displayed in this manner may constitute its own “series,” especially if a single letter is used for each size (i.e., for each tested visual acuity).

Although optotype letters are shown in FIG. 3 (and the other figures), one having skill in the art will recognize that this invention may use any type of symbol or optotype other than letters. Letters, however, may be advantageous because of their widespread use and acceptance as a trusted measure of visual acuity. Further, many patients are used to identifying letters as part of an eye exam, and this feeling of familiarity may make the testing process easier for the practitioner to explain.

In FIG. 3, the display may make one or more sounds upon display of an optotype. For instance, a “beep” or “tone” may be played at the moment an optotype letter is first displayed. This sound may continue for the entire time the letter is on the display, or it may only occur at the beginning (and/or end). The sound may reinforce the patient's understanding that a response is needed. Further, if a patient hears the sound and sees absolutely nothing, the patient himself may come to realize that he may be experiencing at least some loss in visual acuity.

The display of FIG. 3 may be generated in any number of ways. In one embodiment, the display may be part of a stand-alone testing unit. Such a unit may include a screen or a projector with a projection screen. The screen may be a monitor such as a computer monitor or a television monitor. The stand-alone unit may include a microprocessor or other suitable mechanism for executing instructions that generate the moving fixation target and optotypes. In one embodiment, the display may accordingly be generated by one or more computer programs. The computer programs may be written in any suitable language, including but not limited to C, C++, Java, Fortran, Pascal, Basic, Visual Basic, or the like. Additionally, the computer program may be generated by any number of commercial applications that facilitate the generation of graphical displays. For instance, one may use the FLASH suite of programs commercially available from MACROMEDIA, INC. (San Francisco, Calif.) to generate a program suitable for carrying out embodiments described herein. As is known in the art, computer generated optotype letters (or other symbols) of proper size may be generated by use of appropriate fonts and font sizes.

Any one of an innumerable number of response-gathering techniques may be used to keep track of a patients answers to the display of FIG. 3 (and of the other figures). For instance, in one embodiment a practitioner may simply write down a patient's responses on paper in a suitable chart. Alternatively, the responses of a patient may be recorded electronically. Using speech recognition software, one embodiment may not only record a patient's response, but it may also determine if the patient got the “right” response. Accordingly, the program itself may keep track of the patient's score and output for the practitioner a suitable report.

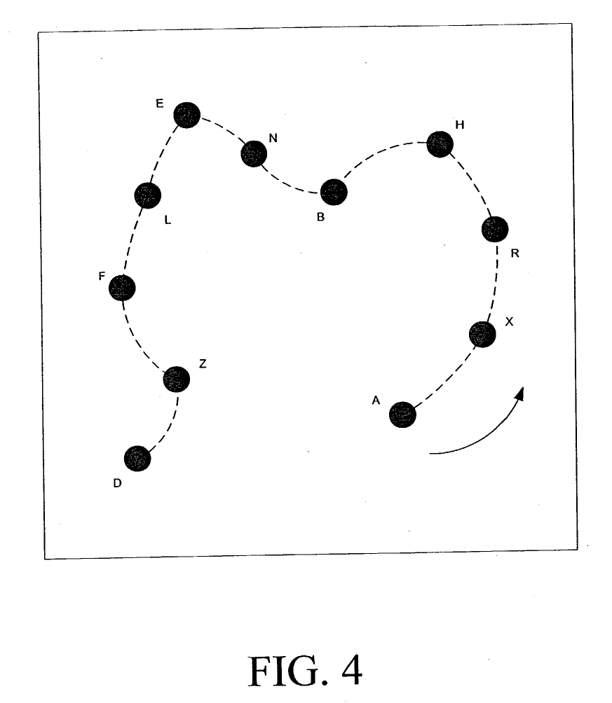

Turning next to FIG. 4, there is a shown a schematic diagram illustrating another embodiment of the OKAy testing scheme. In this test, letters corresponding to an acuity different than that of FIG. 3 are used (the letters in FIG. 4 are smaller than those in FIG. 3).

The description of FIG. 3 is applicable to FIG. 4. The difference in the two figures is that the optotype letters of FIG. 4 correspond to a different acuity. For instance, the letters of FIG. 3 may correspond to 20/200 while the letters of FIG. 4 correspond to 20/100. To access the visual acuity test of FIG. 3, one may press the leftmost square of FIG. 2, while to access the visual acuity test of FIG. 4, one may press the square immediately to its right.

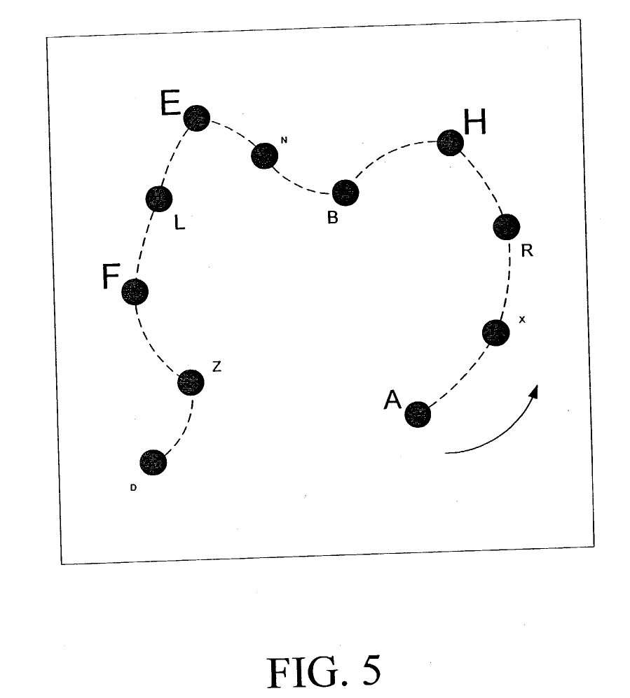

Turning next to FIG. 5, there is a shown a schematic diagram illustrating another embodiment of the OKAy testing scheme. In this test, letters corresponding to mixed acuities are used (the letters in FIG. 5 come in several different sizes, each size corresponding to a particular acuity).

The description of FIG. 3 (and FIG. 4) is applicable to FIG. 5. The difference in is that the optotype letters of FIG. 5 correspond to a variety of different acuities. For instance, some of the letters of FIG. 5 may correspond to 20/200 while other letters may correspond to 20/100, 20/70, 20/50, 20/40, 20/20, 20/15, and the like. To access the visual acuity test of FIG. 5, one may press one of squares of FIG. 2 or load the test in a different manner.

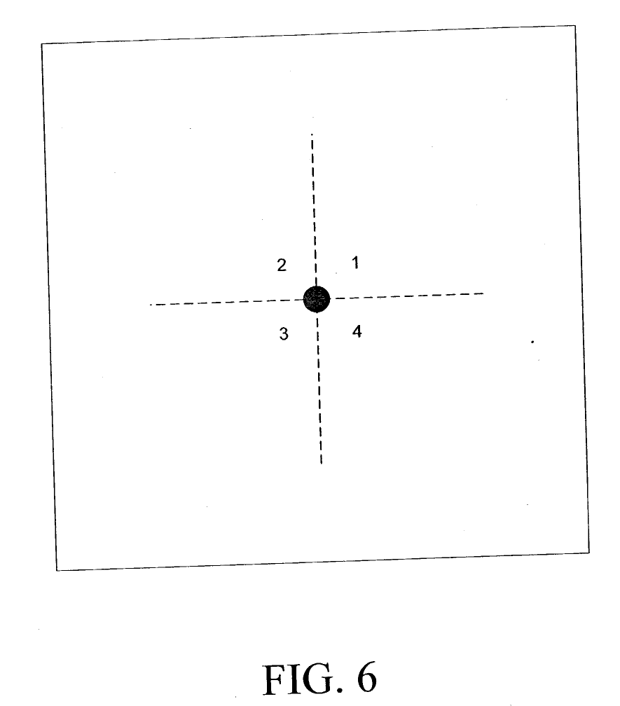

Turning next to FIG. 6, there is a shown a schematic diagram illustrating four quadrants within which an optotype may be placed in accordance with embodiments of the present disclosure.

In FIG. 6, the four quadrants are labeled 1, 2, 3, and 4. In FIGS. 2-5 and 7, the near corner of each optotype is placed about 1.5 degrees from the moving fixation target. In other embodiments, the optotype may be placed closer or farther apart. For instance, the near corner of each optotype may be placed about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 degrees from the moving fixation target. In yet other embodiments, this figure may be even larger or smaller, according to need and to the particular application.

In one embodiment, optotypes may be placed randomly into one of the four quadrants. Using this random approach may lead to, for instance, a first optotype letter being placed in quadrant 1, with subsequent letters being placed in quadrants 2, 1, 4, 3, 2, 1, 3, 2, 2, 4, 1, 2, 3, 4, 3 etc. (i.e., they are placed randomly). This random placement into different quadrants may prevent the patient from being able to “predict” which quadrant an optotype will appear. Correspondingly, the patient may not be able to effectively train his or her microsaccades to compensate for losses in vision and deceptively pass the visual acuity test.

In conjunction with their studies, the inventors have discovered that their OKAy testing scheme can detect scotomata within a region of an eye regardless of which quadrant an optotype is momentarily displayed. Put differently, if a patient has scotomata in a quadrant corresponding to quadrant 1 of FIG. 6, that defect may be detected if an optotype is momentarily displayed in quadrant 1 (for instance, the patient may not be able to identify a letter being displayed, for, for example, 0.1 second in that quadrant). Additionally, and more interestingly, even if an optotype is momentarily displayed in a quadrant other than quadrant 1, that defect may still be detected. Without being bound by theory, the inventors believe this to be the case because such a patient, during testing, is subconsciously engaging in the microsaccades that compensate for loss in visual acuity. Regardless of the quadrant, those microsaccades provide a delay or lag just long enough so that the patient will not be able to reliably identify optotypes placed in different quadrants. This inability, in part, allows the OKAy testing to detect defects that may have gone unnoticed in conventional testing.

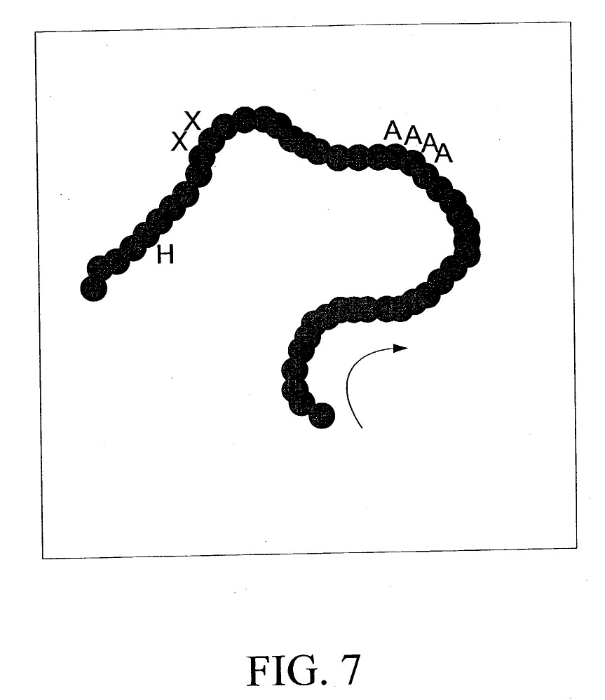

Turning next to FIG. 7, there is a shown a diagram meant to illustrate the motion and display times associated with a visual acuity test using optotype letters, in accordance with embodiments of the present disclosure.

FIG. 7 is meant to show a strobe-type series of snapshots of a display suitable to carry out the OKAy techniques disclosed herein. As illustrated, the “A” optotype letter is displayed twice as long as the “X,” which is displayed twice as long as the “H.” Further, the time period between displaying the “A” and “X” is shorter than the time period between the “X” and the “H.”

With the benefit of the present disclosure, those having skill in the art will comprehend that techniques claimed herein and described above may be modified and applied to a number of additional, different applications, achieving the same or a similar result. The claims attached hereto cover all such modifications that fall within the scope and spirit of this disclosure.

The following examples are included to demonstrate specific embodiments of this disclosure. It should be appreciated by those of skill in the art that the techniques disclosed in the examples that follow represent techniques discovered by the inventors to function well in the practice of the invention, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

The following example summarizes, in abstract form, experiments performed by the inventors. These experiments demonstrate and reinforce the concepts discussed above by utilizing a visual acuity test based on the time-limited display of optotypes offset from a moving fixation target.

Purpose:

To utilize oculokinetic, time limited, offset acuity testing to reveal pericentral scotomata undetectable by standard vision screening tests. Microsaccades allow patients with even large perifoveolar and macular defects to “fill-in” these scotomata and achieve normal vision results on standard acuity or Amsler testing.

Methods:

Twenty-one consenting patients, with an without severe pericentral scotomata by Humphrey threshold perimetry, underwent oculokinetic acuity (OKAy) testing using a moving red-dot fixation target with constant audio feedback. Computer-generated ETDRS letters corresponding to acuities of 20/20, 20/40, 20/50, 20/70, 20/100, and 20/200 were presented in each quadrant with the near corner of each optotype 1.5 degree from fixation, for time intervals of 1.0, 0.5, 0.2, and 0.1 second. Testing proceeded from the largest to smallest optotype size, and from the longest to shortest presentation time for each optotype, in randomized quadrant series.

Results:

The study population included 12 age-matched patients with no pericentral defects (8 female, 4 male, mean age 64.3 years) and 9 patients with dense (>20 dB depression) pericentral defects (4 female, 5 male, mean age 65.9 years) as demonstrated on HVF analysis. Patients without pericentral defects had best-corrected log Mar acuities at 20 ft ranging from 0.4 to 1.0 (mean 0.9+/−sem 0.1), and those with pericentral defects had log Mar acuities ranging from 0.3 to 1.0 (mean 0.7+/−0.1). There was no statistically significant difference in Log Mar acuity between the two groups.

Good correlation (R=0.9) was noted between the standard time-unlimited distance acuity at 20 feet and OKAy acuities at duration 1.0 or 0.5 seconds among all subjects. OKAy testing produced bimodal segregation of patients with pericentral scotomata from those without pericentral defects when offset ETDRS letters were presented for 0.2 or 0.1 seconds. The best intra-test segregation was obtained comparing OKAy results at 0.5 seconds versus 0.1 seconds, which produced consistent acuities in normal eyes, but disparate OKAy acuities (in all quadrants) among subjects with pericentral scotomata.

### CONCLUSION

This study suggests that time limited oculokinetic offset testing at 0.5 seconds can rapidly document standard acuity, and when combined with 0.1 second offset testing, can simultaneously detect pericentral visual defects that elude standard testing strategies. Near or lane-projection OKAy testing may allow for early detection and intervention in patients with pathology leading to pericentral visual field loss from macular degeneration, diabetic retinopathy, and glaucoma.

With the benefit of the present disclosure, those having skill in the art will comprehend that techniques claimed herein may be modified and applied to a number of additional, different applications, achieving the same or a similar result. The claims attached hereto cover all such modifications that fall within the scope and spirit of this disclosure.

### REFERENCES

Each of the following references is hereby incorporated by reference in its entirety:

U.S. Pat. No. 6,313,155

U.S. Pat. No. 6,046,223

U.S. Pat. No. 5,789,435

Damato B E, Chyla J, McClure E, Jay J L, Allen D. A hand-held OKP chart for the screening of glaucoma: preliminary evaluation. Eye 1990;4:632-637.

Mutlukan, E., Damato, B. E. Computerized perimetry with moving and steady fixation in children. Eye 1993, 7: 554-561.

Mutlukan, E., Damato, B. E., Jay, J. F. Clinical evaluation of a multifixation campimeter for the detection of glaucomatous visual field loss. British Journal of Ophthalmology, 1993, 77: 332-338.

Mutlukan, E., Damato, B. E., Tavadia, S. M. Clustering of fixation targets in multifixation campimetry. Eye 1993, 7:131-133.

Mutlukan E., Cullen, J. F. Perimetry Apparatus for the Examination of Central Visual Field, U.K. Patent Office Publications, 9202671.4, London, 1993.

Mutlukan E., Cullen, J. F. Red Colour Comparison Perimetry Chart, U.K. Patent Office Publications, 2247087A, London, 1992.

Cullen J. F., Mutlukan E. Visual field testing for neuro-ophthalmological defects. Optometry Today 1997, 37 (11): 36-37.

Mutlukan, E., Damato, B. E. Evaluation of OKP glaucoma screening chart (Paper), Scottish Ophthalmological Club Meeting. Glasgow, United Kingdom, March 1992.

Mutlukan, E., Stevenson, R. W., Damato, B. E. The oculo-kinetic perimeter in the screening of glaucoma (Paper), College of Ophthalmologists Annual Congress.

Glasgow, U.K., May 1991.

Mutlukan, E. The detection of glaucoma with Oculo-Kinetic Perimetry (Paper), Glaucoma Group of the United Kingdom and Ireland. XIth Annual Meeting. London, United Kingdom, November 1990.

Yamada N, Chen P P, Mills R P, et al. Screening for glaucoma with frequency-doubling technology and Damato campimetry. Arch Ophthalmol 1999;117:1379-1484.

What is claimed is:

1. An apparatus for measuring visual acuity comprising a display configured to show:
(a) a moving fixation target; and
(b) a series of optotypes appearing adjacent the moving fixation target, each optotype appearing only for a limited time.

2. The apparatus of claim 1, wherein the display comprises a projector and a projection screen.

3. The apparatus of claim 1, wherein the display comprises a monitor.

4. The apparatus of claim 1, wherein the moving fixation target comprises a moving circle.

5. The apparatus of claim 1, wherein the moving fixation target tracks a random, continuous path.

6. The apparatus of claim 1, wherein the optotypes comprise letters corresponding to one or more visual acuities.

7. The apparatus of claim 6, wherein the one or more visual acuities comprise 20/20, 20/40, 20/50, 20/70, 20/100, or 20/200.

8. The apparatus of claim 1, wherein each of the optotypes appear in one of four possible random locations adjacent the moving fixation target, each location representing a different quadrant.

9. The apparatus of claim 1, wherein the limited time comprises a time between about 0.05 seconds and about 5 seconds.

10. The apparatus of claim 9, wherein the limited time comprises a time between about 0.1 seconds and about 1 second.

11. The apparatus of claim 10, wherein the limited time comprises about 0.1 seconds, about 0.2 seconds, about 0.5 seconds, or about 1 second.

12. The apparatus of claim 1, wherein different optotypes appear for different times.

13. The apparatus of claim 12, wherein a first optotype appears for about 0.5 seconds and a second optotype appears for about 0.1 seconds.

14. An apparatus for measuring visual acuity comprising a display configured to show:
(a) a moving fixation target; and
(b) a series of optotype letters appearing adjacent the moving fixation target, each optotype letter corresponding to a visual acuity, each optotype letter appearing in one of four possible random quadrant locations adjacent the moving fixation target, and each optotype appearing only for a limited time.

15. The apparatus of claim 14, wherein the display comprises a projector and a projection screen.

16. The apparatus of claim 14, wherein the display comprises a monitor.

17. The apparatus of claim 14, wherein the moving fixation target comprises a moving circle.

18. The apparatus of claim 14, wherein the moving fixation target tracks a random, continuous path.

19. The apparatus of claim 14, wherein the visual acuity comprises 20/20, 20/40, 20/50, 20/70, 20/100, or 20/200.

20. The apparatus of claim 14, wherein the limited time comprises a time between about 0.05 seconds and about 5 seconds.

21. The apparatus of claim 20, wherein the limited time comprises a time between about 0.1 seconds and about 1 second.

22. The apparatus of claim 21, wherein the limited time comprises about 0.1 seconds, about 0.2 seconds, about 0.5 seconds, or about 1 second.

23. The apparatus of claim 14, wherein different optotype letters appear for different times.

24. The apparatus of claim 23, wherein a first optotype letter appears for about 0.5 seconds and a second optotype letter appears for about 0.1 seconds.

25. A computer program for measuring visual acuity comprising:
(a) instructions for displaying a moving fixation target; and
(b) instructions for displaying a series of optotypes appearing adjacent the moving fixation target, each optotype appearing only for a limited time.

26. The computer program of claim 25, wherein each optotype letter corresponds to a visual acuity and each optotype letter appears in one of four possible random quadrant locations adjacent the moving fixation target.

27. A method for measuring visual acuity, comprising:
(a) displaying a moving fixation target; and
(b) displaying a series of optotypes appearing adjacent the moving fixation target, each optotype appearing only for a limited time.

28. The method of claim 27, wherein the moving fixation target comprises a moving circle.

29. The method of claim 27, wherein the moving fixation target tracks a random, continuous path.

30. The method of claim 27, wherein the optotypes comprise letters corresponding to one or more visual acuities.

31. The method of claim 30, wherein the one or more visual acuities comprise 20/20, 20/40, 20/50, 20/70, 20/100, or 20/200.

32. The method of claim 27, wherein each of the optotypes appear in one of four possible random locations adjacent the moving fixation target, each location representing a different quadrant.

33. The method of claim 27, wherein the limited time comprises a time between about 0.05 seconds and about 5 seconds.

34. The method of claim 33, wherein the limited time comprises a time between about 0.1 seconds and about 1 second.

35. The method of claim 34, wherein the limited time comprises about 0.1 seconds, about 0.2 seconds, about 0.5 seconds, or about 1 second.

36. The method of claim 27, wherein different optotypes appear for different times.

37. The method of claim 36, wherein a first optotype appears for about 0.5 seconds and a second optotype appears for about 0.1 seconds.

38. The method of claim 27, wherein the method is performed to show compliance with one or more Food and Drug Administration (FDA) guidelines.

39. A method for measuring visual acuity, comprising:
(a) displaying a moving fixation target; and
(b) displaying a series of optotype letters appearing adjacent the moving fixation target, each optotype letter corresponding to a visual acuity, each optotype letter appearing in one of four possible random quadrant locations adjacent the moving fixation target, and each optotype appearing only for a limited time.

40. The method of claim 39, wherein the moving fixation target comprises a moving circle.

41. The method of claim 39, wherein the moving fixation target tracks a random, continuous path.

42. The method of claim 39, wherein the visual acuity comprises 20/20, 20/40, 20/50, 20/70, 20/100, or 20/200.

43. The method of claim 39, wherein the limited time comprises a time between about 0.05 seconds and about 5 seconds.

44. The method of claim 43, wherein the limited time comprises a time between about 0.1 seconds and about 1 second.

45. The method of claim 44, wherein the limited time comprises about 0.1 seconds, about 0.2 seconds, about 0.5 seconds, or about 1 second.

46. The method of claim 39, wherein different optotype letters appear for different times.

47. The method of claim 46, wherein a first optotype letter appears for about 0.5 seconds and a second optotype letter appears for about 0.1 seconds.

48. The method of claim 39, wherein the method is performed to show compliance with one or more Food and Drug Administration (FDA) guidelines.
